(19) United States
(12) Patent Application Publication
(10) Pub. No.: US 2012/0109190 A1
(43) Pub. Date: May 3, 2012

(54) DEVICES, SYSTEMS, AND METHODS FOR CLOSING AN APERTURE IN A BODILY TISSUE

(75) Inventors: Ghassan S. Kassab, Zionsville, IN (US); Jose A. Navia, SR., Buenos Aires (AR)

(73) Assignee: CVDevices, LLC (a California limited liability company), Zionsville, IN (US)

(21) Appl. No.: 13/102,371

(22) Filed: May 6, 2011

Related U.S. Application Data
(60) Provisional application No. 61/332,029, filed on May 6, 2010.

(51) Int. Cl. A61B 17/03

(52) U.S. Cl. 606/213

(57) ABSTRACT

Devices, systems, and methods for closing an aperture in a bodily tissue. In at least one exemplary embodiment of an aperture closure device of the present disclosure, the aperture closure device comprises an elongated body extending from a proximal end to a distal end, and a closure portion coupled to the elongated body, the closure portion configured to fit within an aperture of a bodily tissue and comprising a material capable of autoexpansion within said aperture.

### PRIORITY

The present application is related to, and claims the priority benefit of, U.S. Provisional Patent Application Ser. No. 61/332,029 filed May 6, 2010, the contents of which are hereby incorporated in their entirety into the present disclosure.

### BACKGROUND

Various medical procedures, especially those involving devices which create apertures/holes within the body, can cause complications in connection with bodily fluids escaping from one part of the body, through an aperture, into another part of the body. For example, various heart procedures that involve the use of wires, needles, catheters, etc., to puncture heart tissue can result in apertures that cause blood to flow from one part of the heart to another, or from one part of the heart to the outside of the heart, including, for example, the pericardial space.

In such situations, it would be desirable to have an effective means for closing the apertures to prevent unwanted fluid flow and to also promote tissue healing. In addition, and in situations where devices remain within the body for an extended period of time (such as with pacing leads), it would be desirable to have effective means for preventing unwanted fluid flow within a tissue aperture having at least a portion of a device positioned therein.

### BRIEF DESCRIPTION

In at least one exemplary embodiment of an aperture closure device of the present disclosure, the aperture closure device comprises an elongated body extending from a proximal end to a distal end, and a closure portion coupled to the elongated body, the closure portion configured to fit within an aperture of a bodily tissue and comprising a material capable of autoexpansion within said aperture to occlude said aperture. In another embodiment, the closure portion comprises a material selected from the group consisting of expanded polytetrafluoroethylene, a dehydrated protein matrix, and polyethylene terephthalate, or a combination thereof. In yet another embodiment, the closure portion is capable of absorbing a bodily fluid to cause the closure portion to expand. In an additional embodiment, the closure portion is capable of autoexpanding by absorbing blood to occlude the aperture. In various embodiments, the closure portion is an autoexpandable sponge-like portion capable of receiving a bodily fluid.

In at least one exemplary embodiment of an aperture closure device of the present disclosure, the aperture closure device further comprises at least one marker positioned at an end of the closure portion. In another embodiment, the at least one marker comprises a first marker and a second marker, wherein the first marker is positioned at or near a first end of the closure portion, and wherein the second marker is positioned at or near a second end of the closure portion. In yet another embodiment, the at least one marker is detectable using a marker detection device. In an additional embodiment, the at least one marker comprises a platinum electrode, and wherein the marker detection device comprises a fluoroscopy device. In yet an additional embodiment, the at least one marker is radiopaque.

In at least one exemplary embodiment of an aperture closure device of the present disclosure, the elongated body comprises a wire. In an additional embodiment, the aperture closure device comprises a pacing lead. In yet an additional embodiment, the aperture closure device further comprises a lead tip positioned at or near the distal end of the elongated body. In another embodiment, the lead tip is capable of engaging cardiac tissue to facilitate heart function.

In at least one exemplary embodiment of an aperture closure device of the present disclosure, the elongated body comprises a first catheter having a lumen therethrough. In another embodiment, the lumen is configured to receive at least part of an apparatus therein. In an additional embodiment, the apparatus is selected from the group consisting of a catheter and a wire. In yet an additional embodiment, the apparatus is a delivery catheter capable of delivering a substance to a location within a mammalian body.

In at least one exemplary embodiment of an aperture closure device of the present disclosure, the closure portion is removably coupled to the elongated body. In another embodiment, the closure portion is biodegradable.

In at least one exemplary embodiment of an aperture closure device of the present disclosure, the aperture closure device comprises an elongated body extending from a proximal end to a distal end, and a closure portion coupled to the elongated body, the closure portion configured to fit within an aperture of a bodily tissue, comprising a material capable of autoexpansion within said aperture, and capable of absorbing a bodily fluid during the autoexpansion, a first marker is positioned at or near a first end of the closure portion, and a second marker is positioned at or near a second end of the closure portion.

In at least one exemplary embodiment of an aperture closure system of the present disclosure, the system comprises any number of aperture closure devices of the present disclosure and a sleeve positioned around at least a portion of the aperture closure device, the elongated sleeve slidingly engaging the aperture closure device.

In at least one exemplary embodiment of a method for closing an aperture in a mammalian tissue of the present disclosure, the method comprises the steps of inserting at least part of an aperture closure device into a mammalian body, and positioning a closure portion of the aperture closure device within an aperture in the mammalian body, wherein the closure portion is capable of closing the aperture. In another embodiment, the method further comprises the step of retracting a sleeve positioned around the closure portion to expose the closure portion. In yet another embodiment, the step of retracting the sleeve allows the closure portion to expand from a compressed configuration. In at least one exemplary embodiment of a method for closing an aperture in a mammalian tissue of the present disclosure, the method further comprises the step of withdrawing a portion of the closure device from the mammalian body.

In an additional embodiment, the step of withdrawing is performed by disconnecting the portion of the closure device from the closure portion so that the closure portion remains within the aperture in the mammalian body. In another embodiment, the closure portion is capable of absorbing a bodily fluid to cause the closure portion to expand. In yet another embodiment, the closure portion is capable of autoexpanding by absorbing blood during autoexpansion to occlude the aperture. In an additional embodiment, the closure portion is capable of autoexpanding to close the aperture. In various embodiments, the closure portion is an autoexpandable sponge-like portion capable of receiving a bodily fluid.

### BRIEF DESCRIPTION OF THE DRAWINGS

FIG. 1A shows an exemplary aperture closure device according to at least one embodiment of the present disclosure;

FIG. 1B shows an exemplary aperture closure device positioned within a tissue aperture, according to at least one embodiment of the present disclosure

FIGS. 2A and 2B show an exemplary aperture closure system according to at least one embodiment of the present disclosure;

FIG. 3 shows an exemplary aperture closure device positioned within a heart, according to at least one embodiment of the present disclosure;

FIG. 4 shows steps of exemplary method of using an aperture closure device, according to at least one embodiment of the present disclosure;

FIGS. 5A, 5B, and 5C show the progression of biodegradation of a closure portion of an aperture closure device, according to at least one embodiment of the present disclosure; and

FIG. 6A shows a marker detection device useful to detect markers of an aperture closure device, according to at least one embodiment of the present disclosure.

### DETAILED SUMMARY

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

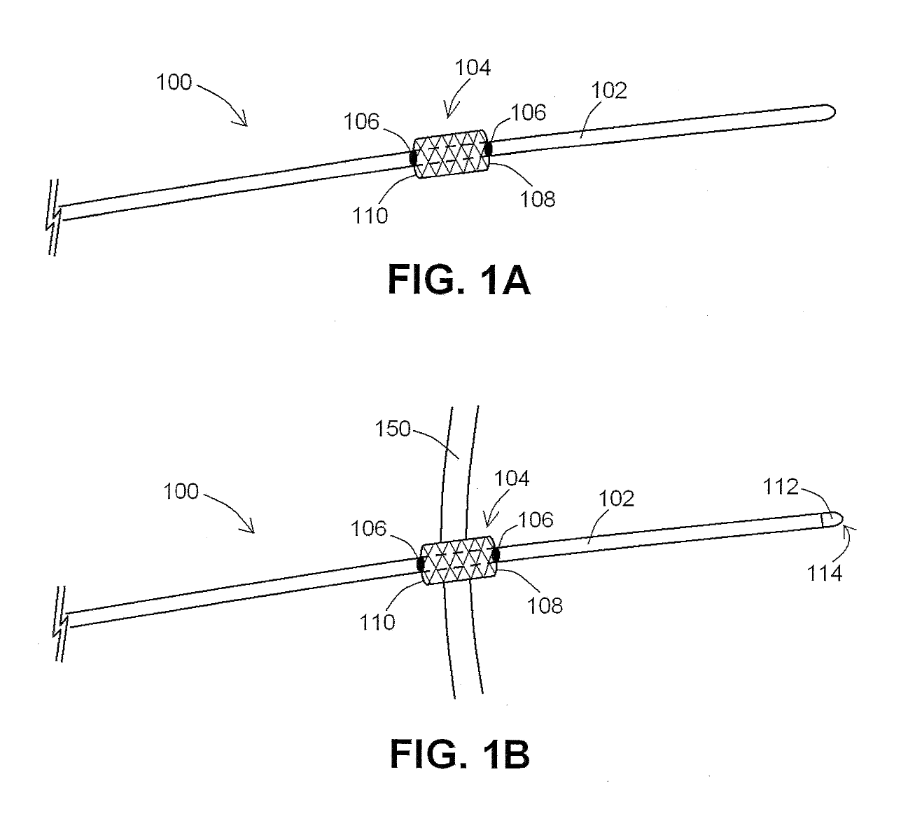

An exemplary embodiment of an aperture closure device of the present disclosure is shown in FIG. 1A. As shown in FIG. 1A, aperture closure device 100 comprises an elongated body 102 and a closure portion 104 positioned thereon. Aperture closure device 100 itself may comprise any number of devices suitable for insertion into a mammalian bodily aperture, including, but not limited to, various catheters, wires, and pacing leads.

In at least one embodiment of an aperture closure device 100 of the present disclosure, closure portion 104 comprises expanded polytetrafluoroethylene (ePTFE), which is a woven polymer consisting of fibrils that are connected by way of notes of polytetrafluoroethylene (PTFE) to create a mesh-like structure. In another embodiment, closure portion 104 may comprise a dehydrated protein matrix (an ameroid), and/or polyethylene terephthalate (PET or PETE), also known as Dacron, either alone or in addition to the ePTFE. Additional closure portions 104 of the present disclosure may comprise any number of biologically compatible sponges or other materials capable of receiving blood and/or other fluids as referenced herein.

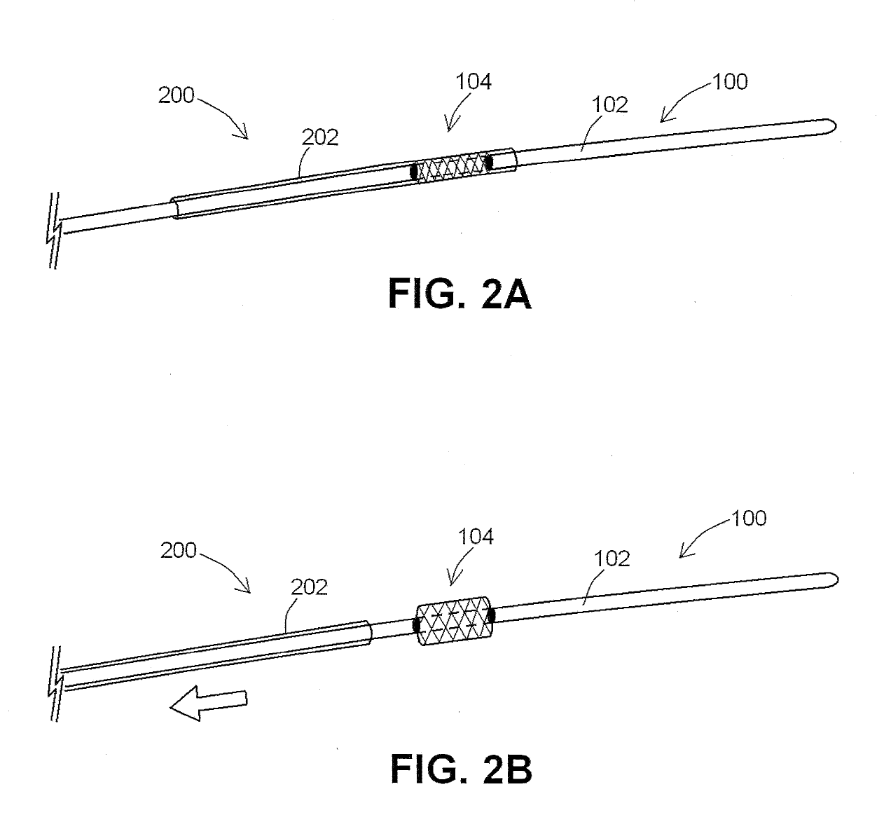

Closure portions 104 of the various embodiments of aperture closure devices 100 of the present disclosure are capable of insertion into a mammalian bodily aperture, either directly (as shown in FIG. 1B) or indirectly by way of a sleeve 202 as shown in FIGS. 2A and 2B. Insertion of closure portion 104 into a bodily aperture, as referenced within the various methods of the present disclosure, closes the bodily aperture as bodily fluids, such as blood, begin to enter closure portion 104 causing closure portion 104 absorb the fluid and potentially causing closure portion 104 to expand. For example, and as referenced in further detail herein regarding an exemplary cardiac application, closure portion 104 may be positioned within a cardiac tissue aperture, and blood may enter closure portion 104 causing closure portion 104 to swell to effective close the aperture. Blood coagulation may further facilitate closure of the aperture when closure portion 104 of device 100 is positioned therein. Autoexpansion, in various embodiments, may be on the order of 1-2 mm, and more or less depending on application.

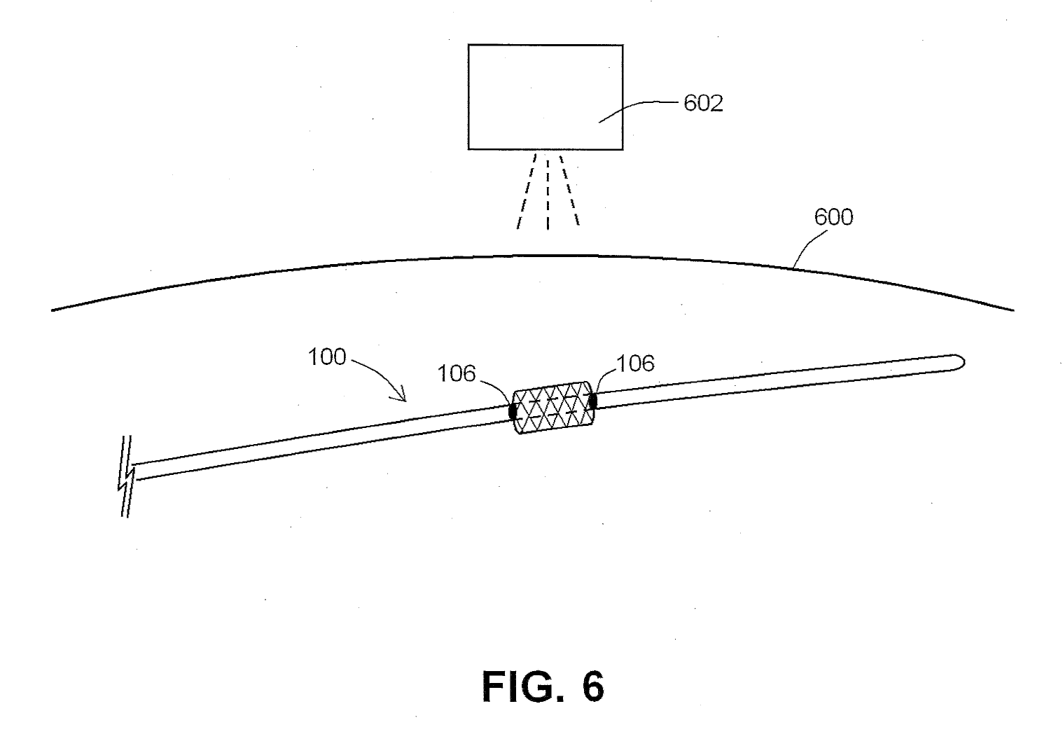

As shown in FIG. 1A, aperture closure device 100 may further comprise one or more markers 106 visible using a marker detection device 602 (not shown in FIG. 1A but shown in FIG. 6). For example, and in at least one embodiment, markers 106 may comprise platinum electrodes, and marker detection device 602 (as shown in FIG. 6) may be a fluoroscopy device capable of detecting the platinum electrodes under fluoroscopy. As shown in FIG. 1A, markers 106 may be positioned at or near one or both of the relative ends (distal end 108 and proximal end 110) of closure portion 104 to facilitate placement of aperture closure device 100 within a body as referenced herein. Various other radioopaque markers, for example, may be used as markers 106 of the present disclosure. In at least one embodiment, closure portion 104 is approximately 1 cm in length (from distal end 108 to proximal end 110).

In an least one embodiment of an aperture closure device 100 of the present disclosure, and as shown in FIG, 1B, elongated body 102 comprises a pacing lead. In such an embodiment, for example, the pacing lead (as described in further detail herein) comprises a lead wire (an exemplary elongated body 102) and a lead tip 112 for engaging cardiac tissue (to facilitate heart function) at or near the distal end 114 of elongated body 102 as shown in FIG. 1B. In various other embodiments of aperture closure devices 100 of the present disclosure, such aperture closure devices 100 do not have a lead tip 112 at or near a distal end 114 of said devices 100.

In FIG. 1B, aperture closure device 100 is shown positioned within an aperture of a bodily tissue 150, whereby the closure portion 104 is positioned at the aperture. As shown in FIG. 1B, one marker 106 is positioned on one side of tissue 150, and another marker 106 is positioned on the other side of tissue 150. Such positioning, for example, may be made under fluoroscopy whereby the placer of aperture closure device 100 within tissue 150 can effectively visualize the location of closure portion 104 with respect to the aperture within tissue 150 to be closed using aperture closure device 100.

FIGS. 2A and 2B show exemplary embodiments of aperture closure systems 200 of the present disclosure. As shown in FIGS. 2A and 2B, aperture closure systems 200 comprise aperture closure devices 100 having an elongated body 102 and a closure portion 104. In FIG. 2A, aperture closure system 200 is shown with a sleeve 202 slidingly engaging aperture disclosure device 100, whereby sleeve 202, when positioned around closure portion 104, compresses closure portion 104 to facilitate, for example, insertion of at least part of aperture closure system 200 into a mammalian body and to position closure portion 104 at a tissue aperture as generally referenced herein. After closure portion 104 is positioned at the tissue aperture, for example, sleeve 202 may be retracted from closure portion 104 (as shown by the arrow in FIG. 2B) to allow closure portion 104 of aperture closure device 100 to decompress/expand and close the aperture as referenced herein.

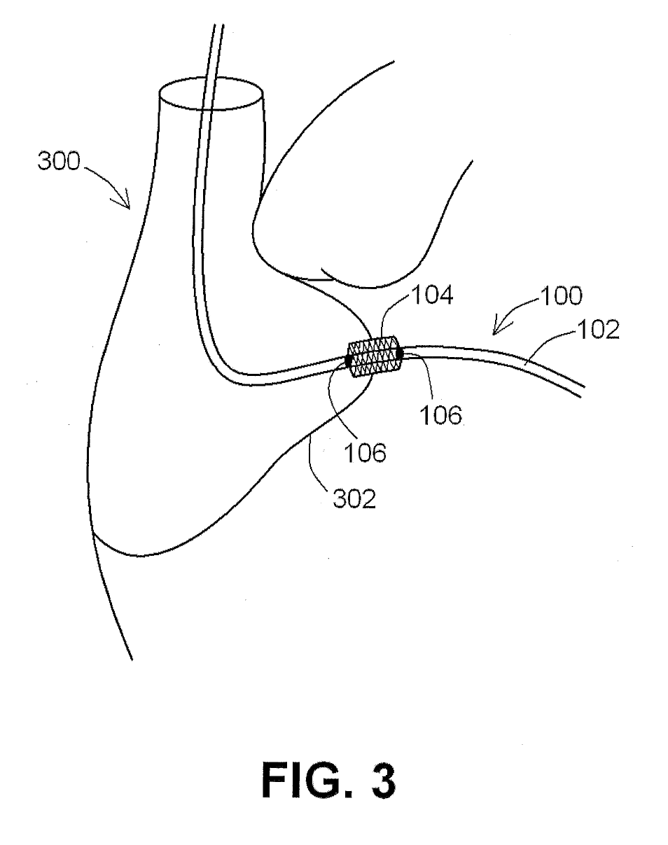

An exemplary embodiment of at least part of an aperture closure device 100 of the present disclosure positioned within a mammalian heart is shown in FIG. 3. As shown in FIG. 3, aperture closure device is shown positioned within an aperture of an atrial wall 302 of a heart 300, whereby closure portion 104 of aperture closure device 100 is posititioned within said aperture. In an embodiment where aperture closure device 100 comprises a pacing lead, for example, a distal end 114 of aperture closure device 112 (as shown in FIG. 1B) may contact heart 300 tissue to assist a pacemaker (not shown) with heart 300 stimulation.

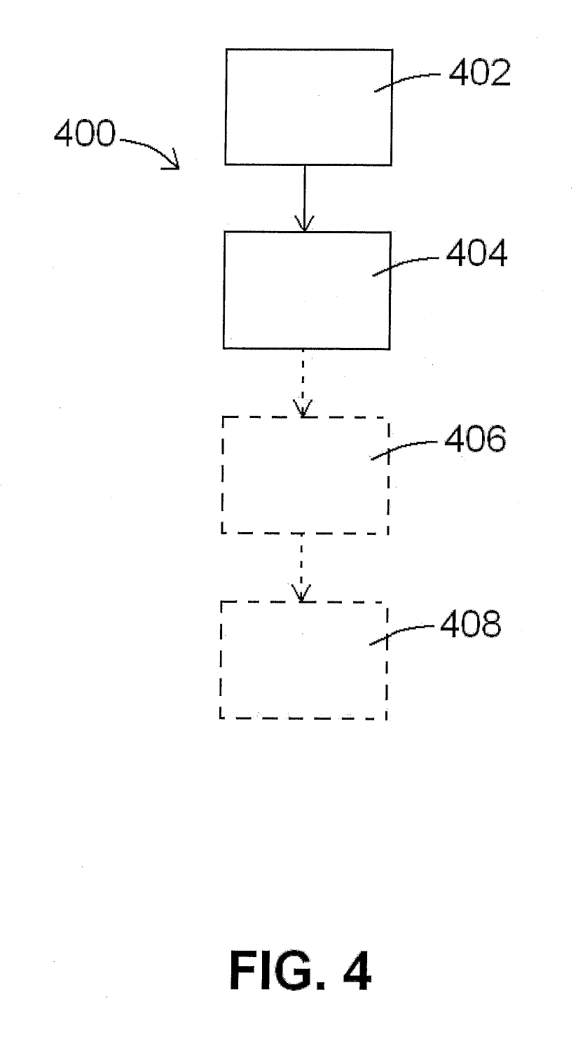

Steps of an exemplary method for closing a bodily aperture using an aperture closure device 100 or an aperture closure system 200 of the present disclosure are shown in FIG. 4. As shown in FIG. 4, method 400 comprises the steps of inserting at least part of an aperture closure device 100 (or an aperture closure system 200) into a body (an exemplary bodily insertion step 402), and positioning a closure portion 104 of aperture closure device 100 within a bodily aperture (an exemplary device positioning step 404). Upon insertion of at least part of device 100 or system 200 into the body, bodily fluids such as blood, for example, may begin to enter closure portion 104 causing closure portion 104 absorb the fluid and potentially causing closure portion 104 to expand. In at least one embodiment, closure portion 104 comprises an autoexpandable sponge-like device, whereby bodily fluids, such as blood, may begin to enter the sponge cells of closure portion 104. For example, and in a cardiac application, closure portion 104 may be positioned within a cardiac tissue aperture in step 404, and blood may enter closure portion 104 causing closure portion 104 to swell to effective close the aperture. Blood coagulation may further facilitate closure of the aperture when closure portion 104 of device 100 is positioned therein.

In an exemplary method 400 of the present disclosure where a system 200 is used, method 400 may further comprise the step of retracting sleeve 202 from closure portion 104 to allow closure portion 104 to decompress/expand to close the aperture (an exemplary sleeve retraction step 406). As referenced herein, and in an exemplary embodiment of a method 400 of the present disclosure, method 400 may further comprise the step of withdrawing the remainder of device 100/system 200 while permitting at least closure portion 104 to remain within the bodily aperture (an exemplary withdrawal step 408).

In at least one embodiment of an aperture closure device 100/aperture closure system 200 of the present disclosure, closure portion 104 is removable from the remainder of device 100/system 200. In such an embodiment, for example, closure portion 104 of device 100/system 200 may be positioned within a tissue aperture, and elongated body 102 may be withdrawn from closure device 104 by way of, for example, pressure from the tissue holding the closure portion 104 in place. In at least another embodiment, closure portion 104 may be detached from elongated body 102 by way of, for example, an internal screw mechanism, one or more magnets, one or more snaps, or other means for generally detaching one portion of a medical device from another as known in the art.

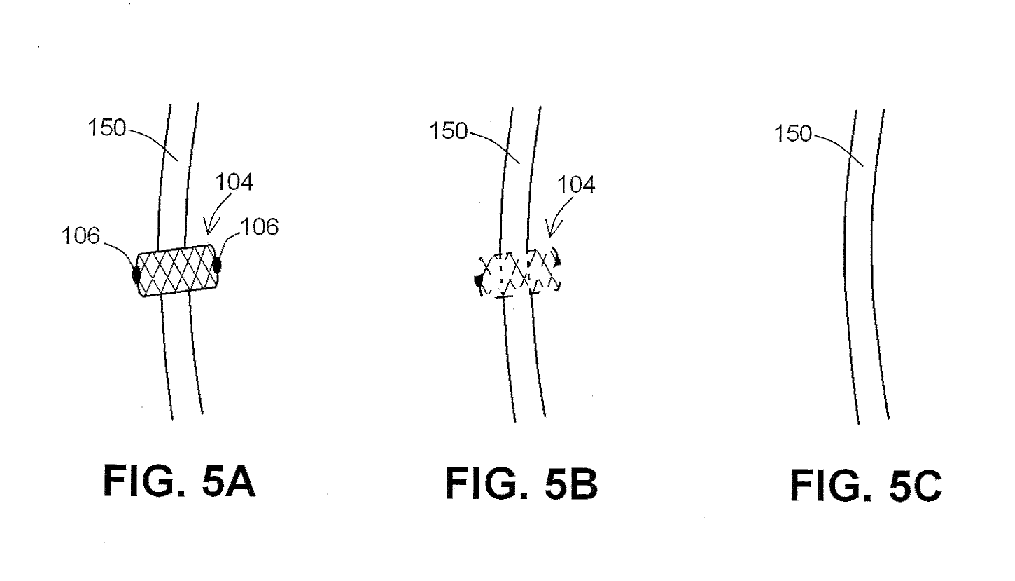

For example, and as shown in FIG. 5A, closure portion 104 is positioned within an aperture of a tissue 150, whereby one marker 106 is on one side of tissue 150, and another marker 106 is on the other side of tissue 150. Closure portion 104, in at least one embodiment, may biodegrade over time (shown as partially degraded in FIG, 5B) until closure portion 104 is completely dissolved and tissue 150 is “healed” (aperture is closed) as shown in FIG. 5C.

FIG. 6 shows an embodiment of an aperture closure device 100 of the present disclosure present within a body. As shown in FIG. 6, at least part of aperture closure device 100 is below the surface of a body 600 (within a body 600), whereby markers 106 of aperture closure device 100 are detectable by way of a marker detection device 602. As referenced herein, a placer of aperture closure device 100 within a body may more precisely position a closure portion 104 of said device 100 within an aperture if the placer can “see” markers 106 by way of marker detection device 602.

While various embodiments of systems and methods for closing an aperture in a bodily tissue have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. An aperture closure device, comprising:
an elongated body extending from a proximal end to a distal end; and
a closure portion coupled to the elongated body, the closure portion configured to fit within an aperture of a bodily tissue and comprising a material capable of autoexpansion within said aperture to occlude said aperture.

2. The aperture closure device of claim 1, wherein the closure portion comprises a material selected from the group consisting of expanded polytetrafluoroethylene, a dehydrated protein matrix, and polyethylene terephthalate, or a combination thereof.

3. The aperture closure device of claim 1, wherein the closure portion is capable of absorbing a bodily fluid to cause the closure portion to expand.

4. The aperture closure device of claim 1, wherein the closure portion is capable of autoexpanding by absorbing blood to occlude the aperture.

5. The aperture closure device of claim 1, wherein the closure portion is an autoexpandable sponge-like portion capable of receiving a bodily fluid.

6. The aperture closure device of claim 1, further comprising:
at least one marker positioned at an end of the closure portion, the at least one marker detectable using a marker detection device.

7. The aperture closure device of claim 6, wherein the at least one marker comprises a first marker and a second marker, wherein the first marker is positioned at or near a first end of the closure portion, and wherein the second marker is positioned at or near a second end of the closure portion,

8. The aperture closure device of claim 6, wherein the at least one marker comprises a platinum electrode, and wherein the marker detection device comprises a fluoroscopy device.

9. The aperture closure device of claim 6, wherein the at least one marker is radiopaque.

10. The aperture closure device of claim 1, wherein the elongated body comprises a wire.

11. The aperture closure device of claim 1, wherein the aperture closure device comprises a pacing lead, further comprising:
a lead tip positioned at or near the distal end of the elongated body, the lead tip is capable of engaging cardiac tissue to facilitate heart function.

12. The aperture closure device of claim 1, wherein the elongated body comprises a first catheter having a lumen therethrough, the lumen is configured to receive at least part of an apparatus therein.

13. The aperture closure device of claim 12, wherein the apparatus is selected from the group consisting of a delivery catheter capable of delivering a substance to a location within a mammalian body and a wire.

14. The aperture closure device of claim 1, wherein the closure portion is removably coupled to the elongated body.

15. The aperture closure device of claim 1, wherein the closure portion is biodegradable.

16. The aperture closure device of claim 1, further comprising:
a sleeve positioned around at least a portion of the aperture closure device, the elongated

17. An aperture closure device, comprising:
an elongated body extending from a proximal end to a distal end; and
a closure portion coupled to the elongated body, the closure portion:
configured to fit within an aperture of a bodily tissue,
comprising a material capable of autoexpansion within said aperture, and
capable of absorbing a bodily fluid during autoexpansion to occlude said aperture;
a first marker is positioned at or near a first end of the closure portion; and
a second marker is positioned at or near a second end of the closure portion;
wherein the first marker and the second marker detectable using a marker detection device.

18. A method for closing an aperture in a mammalian tissue, the method comprising the steps of:
inserting at least part of an aperture closure device into a mammalian body, the aperture closure device comprising:
an elongated body extending from a proximal end to a distal end, and
a closure portion coupled to the elongated body, the closure portion configured to fit within an aperture of a bodily tissue and comprising a material capable of autoexpansion within said aperture; and
positioning the closure portion within the aperture in the mammalian body to close the aperture.

19. The method of claim 18, further comprising the step of:
retracting a sleeve positioned around the closure portion to expose the closure portion.

20. The method of claim 19, wherein the step of retracting the sleeve allows the closure portion to expand from a compressed configuration.

21. The method of claim 18, further comprising the step of:
withdrawing a portion of the closure device from the mammalian body by disconnecting the portion of the closure device from the closure portion so that the closure portion remains within the aperture in the mammalian body.

22. The method of claim 18, wherein the closure portion is capable of autoexpanding by absorbing a bodily fluid during autoexpansion to occlude the aperture.
